(19) United States
(12) Patent Application Publication
(10) Pub. No.: US 2025/0364132 A1
(43) Pub. Date: Nov. 27, 2025

(54) RISK MANAGEMENT SYSTEM, DEVICE, AND RELATED METHODS

(71) Applicant: Bigfoot Biomedical, Inc., Milpitas, CA (US)

(72) Inventor: Corydon A. HINTON, Oakland, CA (US)

(21) Appl. No.: 18/874,990

(22) PCT Filed: Jun. 16, 2023
(86) PCT No.: PCT/US2023/068629

§ 371 Date: Dec. 13, 2024

Related U.S. Application Data
(60) Provisional application No. 63/366,523, filed on Jun. 16, 2022.

(51) Int. Cl. G16H 40/67

(52) U.S. Cl. CPC G16H 40/67

(57) ABSTRACT

The present disclosure is directed to risk management systems, devices, and methods for analyzing and identifying operational deviations of client devices, medical devices, and applications thereon. The systems and methods may receive operational data of a medical device or an application of a client device. The systems and methods may analyze the operational data to identify one or more deviations from an intended operation of the medical device or the application. The systems and methods may generate a notification indicating the one or more deviations, responsive to identifying the one or more deviations. The systems and methods may also provide the notification to one or more of the medical devices or the client device.

### PRIORITY CLAIM

This application claims the benefit of the filing date of U.S. Provisional Patent Application Ser. No. 63/366,523, filed Jun. 16, 2022, for “RISK MANAGEMENT SYSTEM, DEVICE, AND RELATED METHODS.” the disclosure of which is hereby incorporated herein in its entirety by this reference.

### TECHNICAL FIELD

Embodiments discussed herein relate, generally, to a risk management system. More specifically, embodiments relate systems, devices, and methods for analyzing and identifying operational deviations of client devices, medical devices, and applications thereon.

### BACKGROUND

Many people have medical conditions that require regular care and attention. For example, diabetes mellitus is a chronic metabolic disorder caused by an inability of a person's pancreas to produce sufficient amounts of the hormone, insulin, such that the person's metabolism is unable to provide for the proper absorption of sugar and starch. This failure leads to hyperglycemia, i.e., the presence of an excessive amount of analyte, such as glucose, within the blood plasma. Persistent hyperglycemia has been associated with a variety of serious symptoms and life threatening long-term complications such as dehydration, ketoacidosis, diabetic coma, cardiovascular diseases, chronic renal failure, retinal damage and nerve damages with the risk of amputation of extremities. Self-monitoring of blood glucose and the self-administration of insulin is the typical method for treating diabetes. People with Type I, Type II, or gestational diabetes typically track their blood glucose levels and administer self-treatment to maintain appropriate blood glucose levels. Certain medical devices, such as Blood Glucose Meters. Continuous Glucose Monitors (CGMs), infusion pumps, and injection pens, have been developed to assist with monitoring and treating medical conditions such as diabetes. To facilitate easy monitoring and maintenance, many medical devices designed for medical conditions that require constant attention are also configured to interface with client devices through the use of, for example, an application that may be installed on a client device. Medical devices and associated application may also provide a series of notifications, such as alarms and alerts intended to draw a user's attention to situations related to a user's medical condition, system conditions, and/or other potential issues-and more generally reduce the cognitive burden associated with self-monitoring and self-treatment. These notifications may result in alert-fatigue, which may result in users ignoring alarms or alerts or discontinue use of their medical device, thus reducing the quality of their treatment.

### DISCLOSURE

The various embodiments of the present disclosure provide benefits and/or solve one or more of the foregoing or other problems in the art with systems and methods for creating website. Various embodiments of the present disclosure include a method. The method may include receiving, at a cloud computing platform, operational data of a medical device or an application of a client device, the application being associated with the medical device. The method may additionally include analyzing the operational data to identify one or more deviations from an intended operation of the medical device or the application. The method may also include generating a notification indicating the one or more deviations responsive to identifying the one or more deviations. The method may further include providing the notification to one or more of the medical devices or the client device.

One or more embodiments of the present disclosure include a method of determining operational performance of an application. The method may include receiving, at a cloud computing platform, operational data of the application of a client device in communication with a medical device. The method may also include analyzing, at the cloud computing platform, the operational data. Analyzing the operational data may include identifying one or more trigger conditions, and may also include identifying that one or more response actions to the one or more trigger conditions were not performed properly. The method may further include generating a notification indicating a deviation from an intended operation of the application of the client device or the medical device in response to identifying that the one or more response actions were not performed properly. The method may additionally include providing the notification to the client device.

Various embodiments of the present disclosure include systems and devices, such as a cloud computing platform. The cloud computing platform may include a processor and a memory. The memory may store instructions thereon, that, when executed by the processor, cause the cloud computing platform to perform one or more acts, including receiving operational data, analyzing the operational data, generating a notification, and providing the notification. The cloud computing platform may receive, from a mobile device, operational data of a medical device or an application of the mobile device. The cloud computing platform may analyze the operational data to identify one or more deviations from an intended operation of the medical device or the application. The cloud computing platform may generate a notification indicating the one or more deviations, responsive to identifying the one or more deviations. The cloud computing platform may also provide a notification to the mobile device one or more times according to a determined frequency.

### BRIEF DESCRIPTION OF THE DRAWINGS

To easily identify the discussion of any particular element or act, the most significant digit or digits in a reference number refer to the figure number in which that element is first introduced.

FIG. 1 illustrates a schematic representation of an environment within which a risk management system may operate in accordance with one or more embodiments of the present disclosure:

FIG. 2 illustrates a sequence flow diagram that the risk management system may utilize to provide warning notifications in accordance with one or more embodiments of the present disclosure:

FIG. 3 illustrates a flow diagram that the risk management system may utilize to create one or more response actions in accordance with one or more embodiments of the present disclosure;

FIG. 4 illustrates another flow diagram that the risk management system may utilize to create one or more response actions in accordance with one or more embodiments of the present disclosure; and

FIG. 5 illustrates a block diagram of an example computing device in accordance with one or more embodiments of the present disclosure.

FIG. 6 illustrates a diagram of an example flow for a system that maintains a catalog of platform dependent features, settings, and uses configurable values, and log tags for the same.

FIG. 7 illustrates a diagram of an example flow for a system that analyzes operations logs archived in a DataLake, in accordance with one or more examples.

### MODE(S) FOR CARRYING OUT THE INVENTION

In the following detailed description, reference is made to the accompanying drawings, which form a part hereof, and in which are shown, by way of illustration, specific example embodiments in which the present disclosure may be practiced. These embodiments are described in sufficient detail to enable a person of ordinary skill in the art to practice the present disclosure. However, other embodiments may be utilized, and structural, material, and process changes may be made without departing from the scope of the disclosure.

As used herein, any relational term, such as “first,” “second,” “front,” “back,” etc., is used for clarity and convenience in understanding the disclosure and accompanying drawings, and does not connote or depend on any specific preference or order, except where the context clearly indicates otherwise.

As used herein, the terms “comprising,” “including,” “containing,” “characterized by,” and grammatical equivalents thereof are inclusive or open-ended terms that do not exclude additional, un-recited elements or method steps, but also include the more restrictive terms “consisting of.” “consisting essentially of,” and grammatical equivalents thereof.

As used herein, the term “may” with respect to a material. structure, feature. or method act indicates that such is contemplated for use in implementation of an embodiment of the disclosure, and such term is used in preference to the more restrictive term “is” so as to avoid any implication that other compatible materials, structures, features, and methods usable in combination therewith should or must be excluded.

As used herein, the term “configured” refers to a size, shape, material composition, and arrangement of one or more of at least one structure and at least one apparatus facilitating operation of one or more of the structures and the apparatus in a predetermined way.

As used herein, the singular forms following “a,” “an,” and “the” are intended to include the plural forms as well, unless the context clearly indicates otherwise.

As used herein, the term “and/or” includes any and all combinations of one or more of the associated listed items.

As used herein, the term “substantially” in reference to a given parameter, property, or condition means and includes to a degree that one of ordinary skill in the art would understand that the given parameter, property, or condition is met with a degree of variance, such as within acceptable tolerances. By way of example, depending on the particular parameter, property, or condition that is substantially met, the parameter, property, or condition may be at least 90.0 percent met, at least 95.0 percent met, at least 99.0 percent met, at least 99.9 percent met, or even 100.0 percent met.

As used herein, “about” or “approximately” in reference to a numerical value for a particular parameter is inclusive of the numerical value and a degree of variance from the numerical value that one of ordinary skill in the art would understand is within acceptable tolerances for the particular parameter. For example, “about” or “approximately” in reference to a numerical value may include additional numerical values within a range of from 90.0 percent to 110.0 percent of the numerical value, such as within a range of from 95.0 percent to 105.0 percent of the numerical value, within a range of from 97.5 percent to 102.5 percent of the numerical value, within a range of from 99.0 percent to 101.0 percent of the numerical value, within a range of from 99.5 percent to 100.5 percent of the numerical value, or within a range of from 99.9 percent to 100.1 percent of the numerical value.

The following description may include examples to help enable one of ordinary skill in the art to practice the disclosed embodiments. The use of the term “for example,” means that the related description is explanatory, and though the scope of the disclosure is intended to encompass the examples and legal equivalents thereof. the use of such terms is not intended to limit the scope of an embodiment or this disclosure to the specified components, steps. features, functions, or the like.

It will be readily understood that the components of the embodiments as generally described herein and illustrated in the drawings could be arranged and designed in a wide variety of different configurations. Thus, the following description of various embodiments is not intended to limit the scope of the present disclosure, but is merely representative of various embodiments. While the various aspects of the embodiments may be presented in the drawings, the drawings are not necessarily drawn to scale unless specifically indicated.

Embodiments of the present disclosure may be described in terms of a process that is depicted as a flowchart, a flow diagram, a structure diagram, or a block diagram. Although a flowchart may describe operational acts as a sequential process, many of these acts may be performed in another sequence, in parallel. or substantially concurrently. In addition, the order of the acts may be re-arranged. A process may correspond to a method, a thread, a function, a procedure, a subroutine, a subprogram, other structure, or combinations thereof. Furthermore, the methods disclosed herein may be implemented in hardware, software, or both. If implemented in software, the functions may be stored or transmitted as one or more instructions or code on computer-readable media. Computer-readable media includes both computer storage media and communication media including any medium that facilitates transfer of a computer program from one place to another.

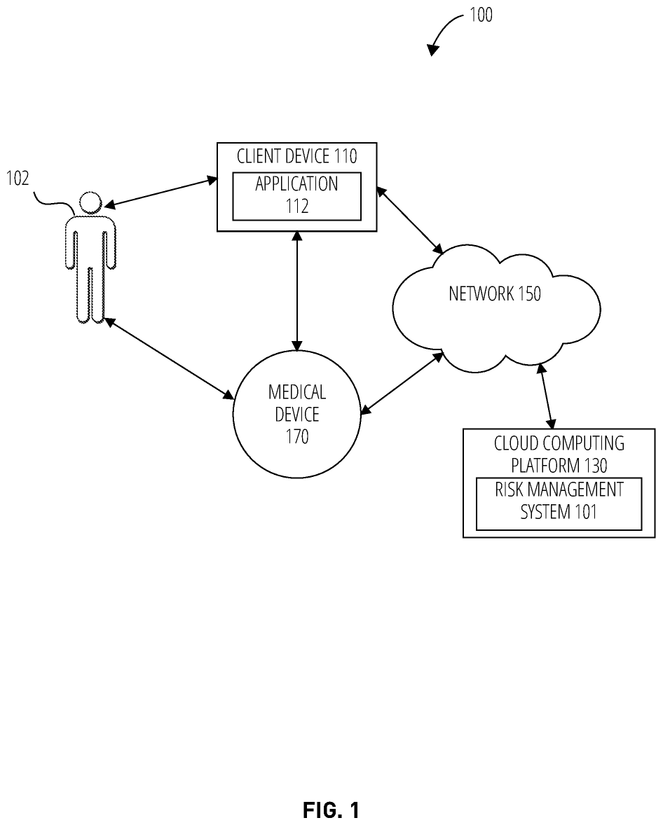

FIG. 1 illustrates a schematic diagram of an environment 100 in which a risk management system 101 may operate according to one or more embodiments of the present disclosure. As illustrated, the environment 100 includes a client device 110, a cloud computing platform 130, a network 150, and a medical device 170. The client device 110, the cloud computing platform 130, and the medical device 170 may communicate via the network 150. The network 150 may include one or more wired or wireless communication networks, including, without limitation the Internet, and may use one or more communications platforms or technologies suitable for transmitting data and/or communication signals (e.g., Ethernet, Bluetooth, Bluetooth Low Energy, Cellular, WiFi, Near Field Communication (NFC), without limitation). Although FIG. 1 illustrates a particular arrangement of the client device 110. the cloud computing platform 130, the medical device 170, and the network 150, various additional arrangements are possible. For example, as illustrated in FIG. 1, the client device 110 may directly communicate with the medical device 170. bypassing the network 150.

In one or more embodiments, a user 102 (e.g., an individual. without limitation) may interact directly with a medical device 170. For example, the medical device 170 may include a CGM, a medication delivery device, such as an infusion pump or injection pen, a pen cap associated with a pen, a physiological sensor for temperature or heart rate, or a combination of any of the foregoing devices. In various embodiments, the user 102 may interact with the client device 110, for example, to communicate with the medical device 170 and/or the cloud computing platform 130. The client device 110 may include an application 112 installed thereon. The application 112 may be associated with the medical device 170 and/or the cloud computing platform 130. For example, the application 112 may support and/or enable the client device 110 to directly or indirectly interface with the medical device 170 and/or the cloud computing platform 130. The application 112 may also gather operational data related to an operation of the medical device 170, the application 112, and/or the client device 110. The client device 110, the application 112, and/or the medical device 170 may be associated with a specific individual user

The application 112 may be is a computer program that performs specific functions discussed herein. The application 112 is associated with the medical device 170. For example, the medical device 170 may be communicatively connected to the client device 110 and/or the application 112 thereon. In one or more embodiments, the medical device 170 may be communicatively connected to the client device 110 and/or the application 112 by a physically connection (e.g., by a wire or cable) or a logical connection to the client device 110 (e.g., via an Ethernet network, without limitation). Additionally or alternatively to physical connections, the medical device 170 may be communicatively connected to the client device 110 and/or the application 112 by a wireless connection (e.g., via Bluetooth®, Wi-Fi. NFC, without limitation). In one or more examples. the application 112 may not be associated with further medical devices when it is associated with medical device 170. The application 112 may track the operation of a specific medical device (e.g., the medical device 170), such as by monitoring and gathering operational data of the medical device 170, as described in further detail below. Additionally, the application 112 may, or may be utilized to, at least partially operate the medical device 170. As a non-limiting example, the application 112 may, or may be utilized to, control the medical device 170 to administer, adjust, re-schedule, and/or cancel a treatment (e.g., a dose of medicine (e.g., insulin, without limitation), without limitation).

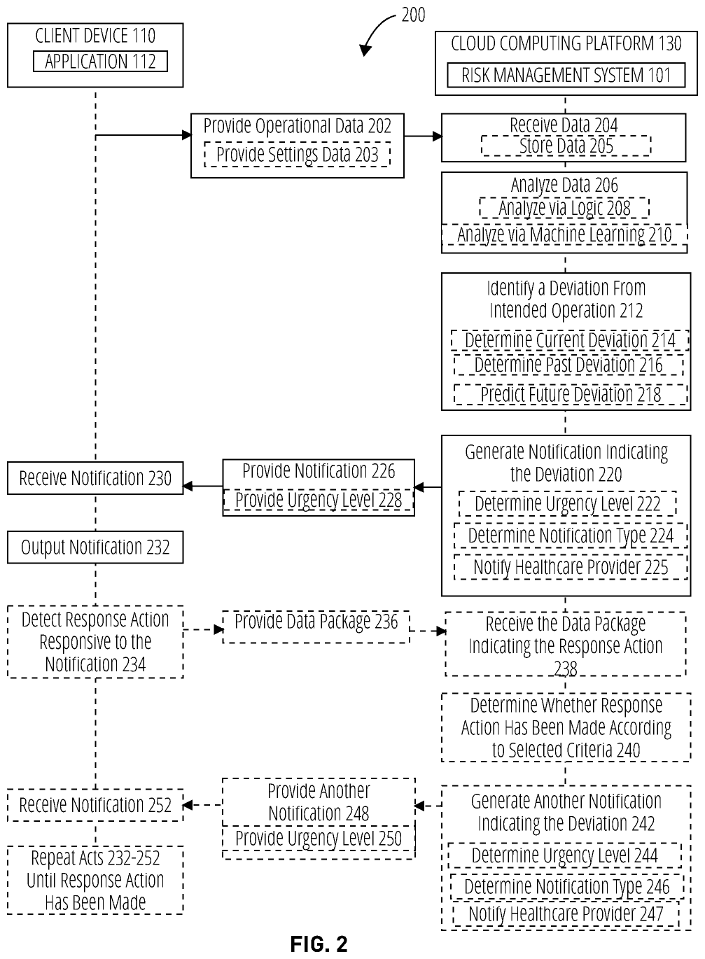

In various embodiments. the cloud computing platform 130 may include the risk management system 101. In one or more embodiments, the risk management system 101 may be associated with the medical device 170, a manufacturer of the medical device 170, and/or a health care provider. In various embodiments, one or more of the manufacturers of the medical device 170 or the health care provider may provide information and/or guidelines related to the appropriate operation (e.g., intended operation) of the medical device 170 and/or the application 112. For example, one or more of the manufacturer of the medical device 170 or the health care provider may provide the cloud computing platform 130 with information related to an intended operation of the medical device 170 and/or the application 112, in addition to information related to intended communication protocol between the risk management system 101, the medical device 170, the application 112, the client device 110, the network 150, and/or the cloud computing platform 130. In various embodiments, the risk management system 101 may analyze data representing operation of the medical device 170 and/or the application 112 to identify deviations from intended operation of the medical device 170 and/or the application 112, as is described in further detail below in regard to FIGS. 2-5.

The client device 110 and the cloud computing platform 130 may represent various types of computing devices with which users may interact. For example, the client device 110 and/or the cloud computing platform 130 may be a mobile device (e.g., a cell phone, a smartphone, a personal data assistant (PDA). a tablet computer, a laptop computer, a desktop computer, a wearable computer or device (e.g., a smart watch, without limitation), etc.). In various embodiments, however, the client device 110 and/or cloud computing platform 130 may include one or more non-mobile computer systems (e.g., a desktop or server, without limitation).

FIG. 2 illustrates a sequence-flow diagram 200 that a risk management system may implement to analyze operation of an application and/or a medical device, and notify a user of a deviation from an intended operation of the application and/or the medical device. As described herein, the sequence-flow diagram 200 may involve the system and/or one or more devices illustrated in FIG. 1, including, for example, the cloud computing platform 130, the risk management system 101, the client device 110, the application 112, the medical device 170, the network 150, and/or the user 102.

Referring to the sequence-flow diagram 200, a client device 110 or the application 112 may track (e.g., gather and/or record) operational data related to an operation of the medical device 170 (FIG. 1) and/or the application 112 associated with the medical device 170 (FIG. 1). For example. the client device 110 may gather the operational data of the medical device 170 (FIG. 1) directly (e.g., via the medical device 170 (FIG. 1)) or indirectly (e.g., via the application 112). In various embodiments. the client device 110 may track the operational data via operational logs and/or other data packages within a database or memory. For example, the operational data may include time-varying data gathered via synchronizing events at periodic intervals (e.g., at least once per hour, at least once per minute, at least once per second, at least once per fraction of a second, etc.). Time-varying operational data may, for example, provide an indication of any changes that have occurred in the operation of the client device 110, the application 112, and/or the medical device 170.

As illustrated in FIG. 2, the client device 110 and/or the application 112 may provide (e.g., send, transmit) the operational data of the medical device 170 (FIG. 1) to the cloud computing platform 130. as shown in act 202. For example, the client device 110 and/or the application 112 may provide operational logs and/or data packages that include operational data to the risk management system 101 of the cloud computing platform 130. In one or more embodiments, providing the operational data to the cloud computing platform 130 may include providing settings data (e.g., current settings, changes to settings, etc.) of the medical device 170, the application 112, and/or the client device 110, as shown in optional act 203 of FIG. 2. For example, the settings data may include current notification settings, network connection settings, and current software versions (e.g., versions of operating systems and/or applications, without limitation). In addition, and without limitation, the settings data may include whether the medical device 170 and/or the client device 110 is in a certain mode such as airplane mode, sleep mode, do not disturb mode, and/or silent mode that may affect notifications. The settings data may further include, without limitation, changes to notification settings. network connection settings. and/or software versions (e.g., software updates). The changes to notification settings and/or network connection settings may occur, for example, in response to user interaction and/or automatically in response to software updates.

The client device 110 may provide the operational data to the cloud computing platform 130 via the application 112 when the client device 110 is connected to a network (e.g., network 150 (FIG. 1)). In one or more embodiments. the client device 110 and/or application 112 may provide the operational data and/or the settings data to the risk management system 101 and cloud computing platform 130 automatically (i.e., in response pre-specified triggers without further user supervision or approval) while the client device 110 is connected to a network (e.g., Wi-Fi). For example, in various embodiments, the client device 110 and/or application 112 may provide the operational data to the cloud computing platform 130 at periodic intervals (e.g., at least one per day, at least once per hour, at least once per minute, at least once per second, at least once per fraction of a second, without limitation) and/or under predefined conditions (e.g., if the client device 110 is connected to a certain type of network such as Wi-Fi, without limitation). That is, the application 112 and/or client device 110 may be configured to at least partially synchronize the operational data and other data based thereon at the risk management system 101 and/or the cloud computing platform 130 with new operational data at periodic intervals and/or under predefined conditions.

In one or more embodiments, the cloud computing platform 130 may receive data (e.g., the operational data and/or the settings data), as shown in act 204 of FIG. 2. For example, the cloud computing platform 130 may receive the operational data through the network 150 (FIG. 1). Receiving the operational data in act 204 may include storing the operational data, as shown in optional act 205 of FIG. 2. Storing the operational data in act 205 may. for example, enable the risk management system 101 to generate a local operations log that includes historical operational data of the client device 110, the application 112, and/or the medical device 170 (FIG. 1). In addition, the local operations log generated by the risk management system 101 may describe instances in which the operational data was expected to be received and indicate whether or not the operational data was received.

Responsive to receiving the operational data of the medical device 170 (FIG. 1) and/or the application 112 in act 204, the risk management system 101 of the cloud computing platform 130 may analyze the operational data. as shown in act 206 of FIG. 2. The risk management system 101 may analyze the operational data in act 206 using rules-based logic, machine learning, and/or algorithms, as shown in optional act 208 of FIG. 2.

In various embodiments, the risk management system 101 may 1) analyze the operational data in the local operations logs in act 206 to identify alarm trigger conditions exhibited therein and to identify timing of response actions relative to the identified alarm trigger conditions. In other words, the risk management system 101 may analyze the operational data as recorded in the local operations logs to identify events (i.e., alarm trigger conditions) predetermined to warrant a response (i.e., response action) from one or more of the medical device 170 (FIG. 1). the application 112. the client device 110, and/or the user 102 (FIG. 1). In further embodiments, the risk management system 101 may 2) analyze the operational data to identify patterns (e.g., historical trends) of alarm trigger conditions and/or critical events within the logs. In various embodiments, the risk management system 101 may 3) analyze the operational data to identify user attributes that may warrant a response action.

In particular, the risk management system 101 may analyze the operational data recorded in the local operations logs to identify operations and/or data associated with alarm trigger conditions. For example, the risk management system 101 may identify certain operations and/or status data of the client device 110 (e.g., battery life, battery health, and/or errors, etc.), the application 112 (e.g., crashes, errors, etc.), and/or the medical device 170 (e.g., battery life, battery health, and/or errors, etc.) that are associated with alarm trigger conditions according to predetermined criteria. Other examples of the alarm trigger conditions include a quantity of medication (e.g., a quantity of insulin, without limitation) remaining in the medical device 170 (FIG. 1), medication dosing times, missed dosing actions, incorrectly administered doses, upcoming dosing actions, without limitation. Further examples of the alarm trigger conditions include physiological conditions of a user, such as, for example, a blood-glucose level, a body temperature, a heart rate, oxygen levels, and/or blood pressure of the user.

In one or more examples, the risk management system 101 may analyze the operational data to identify critical events that may be relevant to medication therapy of the user 102 (FIG. 1). For example, a critical event may include one or more of the client devices 110, the application 112, and/or the medical device 170 (FIG. 1) malfunctioning and/or being inoperable for a non-negligible period of time. As further examples. critical events may include circumstances in which physiological conditions of the user 102 (FIG. 1) greatly deviate from normal levels (e.g., very high blood-glucose levels, very low blood-glucose levels, etc.).

In some instances, the alarm trigger conditions and/or critical events may be pre-determined to warrant one or more response actions performed by one or more of the client devices 110, the application 112, the user 102, and/or the medical device 170 (FIG. 1). For example, the response action may include providing a prompt (e.g., an audible (e.g., a distinctive sound, without limitation), visual (e.g., a blinking light, flashing icon, message, or other visually distinctive effect. without limitation), or physical (e.g., tactile prompt. without limitation)) to the user 102 via one or more of the client device 110, the application 112, and/or the medical device 170 (FIG. 1), and requiring acknowledgment of the prompt via a user interaction to clear the prompt. Additional examples of response actions may include administration of recommended medication dose, connecting one or more of the client devices 110 and the medical device 170 to external power, and/or adjusting a setting (e.g., volume) of one or more of the client devices 110, the application 112, and the medical device 170. Further examples of response actions may include sending a user a communication (an email, phone call, text message, etc.), the user sending a communication, and/or sending a healthcare provide a communication.

Referring still to FIG. 2, based at least partially on the operational data. the risk management system 101 may determine whether an appropriate response action was performed in response to the one or more identified alarm trigger conditions and/or critical events. Furthermore, in various embodiments, the risk management system 101 may determine whether the associated alarm action was performed within a given time frame (e.g., within a predetermined period of time, without limitation) after an occurrence of the one or more identified alarm trigger conditions and/or critical events. As is discussed in further detail below, if the risk management system 101 determines that a response action was not performed. or that the response action was delayed, the risk management system 101 may determine that a deviation from an intended operation has occurred. In various embodiments, the risk management system 101 may determine that settings of the client device 110, the application, and/or the medical device 170 (FIG. 1) may have prevented the response action.

In one or more embodiments. the risk management system 101 may analyze the operational data to identify patterns (e.g., historical trends) associated with alarm trigger conditions and/or critical events exhibited by the operational logs. For example. the risk management system 101 may identify patterns. such as, frequency of occurrence of alarm trigger conditions and/or critical events, the repeating causes of each alarm trigger condition and/or critical event, and/or where each alarm trigger condition and/or critical event originated (e.g., the medical device 170 (FIG. 1). the application 112, and/or the client device 110). The risk management system 101 may identify a pattern associated with alarm trigger conditions and/or critical events for which, repeatedly, no alarm action was performed. The risk management system 101 may identify patterns associated with the time elapsed from identifying the alarm trigger conditions and/or critical events to when the response action was performed.

As is also mentioned above, the risk management system 101 may also analyze the operations logs to determine user attributes reflected in the operations logs. For example, the risk management system 101 may determine one or more of an experience level of a user, a severity of disease of a user, a responsiveness of a user to alarms, without limitation (e.g., determine a value or description that represents the same. without limitation). In various embodiments, user attributes may be attributes that affect whether response actions are warranted in response to alarm trigger conditions and/or critical events. For example, an experience level of the user 102 (FIG. 1) with regard to using the application 112, the client device 110 and/or the medical device 170 (FIG. 1) may influence whether or not an alarm action is required in response to an alarm trigger condition and/or critical event or a number of response actions required in response to an alarm trigger condition and/or critical event. For example, more response actions may be warranted for inexperienced (e.g., less experienced) users. Conversely, fewer response actions may be warranted for experienced (e.g., more experienced) users. The overall health of the user may influence whether an alarm action is warranted in response to an alarm trigger condition and/or critical event. For example, more alarm conditions may be warranted for users who have a number of pre-existing health conditions or a level of diabetes of the user is considered severe, and fewer alarm conditions may be warranted for users who have no pre-existing health conditions or have a less severe case of diabetes.

Responsive to identifying one or more patterns (e.g., historical trends) associated with alarm trigger conditions or critical events, the risk management system 101 may identify a sensitivity factor that may influence a determination and delivery of an ultimate warning or alarm (described below) to the user. In various embodiments. the sensitivity factor may reflect a frequency at which alarm trigger conditions and critical events occur for a given user. For example, a user experiencing multiple or more often critical events or alarm trigger conditions, as reflected in the operations logs, may be assigned a high sensitivity factor. Conversely, a user experience relatively few critical events or alarm trigger conditions, as reflected in the operations logs, may be assigned a high sensitivity factor. As is discussed in further detail below, a user assigned a high sensitivity factor may be more sensitive to disruptions in the application's 112 or the medical device's 170 (FIG. 1) ability to perform critical tasks and may warrant more frequent notifications regarding the ultimate warning or alarm in comparison to a user assigned a low sensitivity factor.

Additionally, based on the user attributes, the risk management system 101 may determine an experience factor, which is a factor that may influence a determination and delivery of an ultimate warning or alarm (described below) to a user. For example, a determined experience factor may be based at least partially on an experience level of the user in using one or more of the applications 112, the client device 110, and/or the medical device 170 (FIG. 1), as reflected in the local operations logs (i.e., operational data). In various embodiments, an experience level of a user using each of the application 112, the client device 110, and/or the medical device 170 is considered in determining the experience factor of the user. As is discussed below, a user having a high experience factor may warrant fewer or less frequent notifications regarding the ultimate warning or alarm in comparison to a user having a low experience factor.

Referring still FIG. 2, in one or more embodiments, analyzing the operational data may include analyzing the operational data via one or more machine learning or artificial intelligence techniques. For example, the risk management system 101 of the cloud computing platform 130 may analyze the operational data using machine learning and/or artificial intelligence techniques. as shown in optional act 210 of FIG. 2.

In various embodiments, the risk management system 101 may analyze the operational data utilizing one or more of regression models (e.g., a set of statistical processes for estimating the relationships among variables), classification models, and/or phenomena models. Additionally, the machine-learning models may include a quadratic regression analysis, a logistic regression analysis, a support vector machine, a Gaussian process regression, ensemble models, or any other regression analysis. Furthermore, in yet further embodiments. the machine-learning models may include decision tree learning. regression trees, boosted trees, gradient boosted tree. multilayer perceptron, one-vs-rest, Naïve Bayes, k-nearest neighbor, association rule learning, a neural network. deep learning, pattern recognition, or any other type of machine-learning.

Continuing with FIG. 2, in response to analyzing the operational data and identifying alarm trigger conditions, the risk management system 101 may identify one or more deviations from an intended operation of the medical device 170 (FIG. 1) and/or an intended operation of the application 112, as shown in act 212 of FIG. 2. For example, the risk management system 101 may identify the one or more deviations based on any identified alarm trigger conditions and/or critical events that were not properly addressed (e.g., that were not properly address via one or more response actions). As mentioned briefly above, identifying the one or more deviations from an intended operation may include identifying a lack of a response action from a user, a delay in a response action relative to an intended response time, too many response actions, too few response actions, etc., for identified alarm trigger conditions and/or critical events. In addition, patterns (e.g., historical trends) in alarm trigger conditions, critical events, and/or response actions identified by the risk management system 101 may indicate one or more deviations from the intended operation. As non-limiting examples, the risk management system 101 may identify one or more deviations by determining an increased frequency of alarm trigger conditions and/or critical events, repeats of the same type of alarm trigger condition and/or critical event, and/or alarm trigger conditions and/or critical events originating from the same source (e.g., the medical device 170 (FIG. 1), the application 112, or the client device 110).

In various embodiments. the risk management system 101 of the cloud computing platform 130 may determine a current (e.g., present, on-going, unresolved, without limitation) deviation from the intended operation of the medical device 170 (FIG. 1) and/or the application 112, as shown in optional act 214 of FIG. 2. In various embodiments, the risk management system 101 determines past (e.g., previous, resolved, etc.) deviations from the intended operation of the medical device 170 (FIG. 1) and/or the application 112, as shown in optional act 216 of FIG. 2. In one or more embodiments, the risk management system 101 predicts one or more future deviations (e.g., expected, anticipated, etc.) from the intended operation of the medical device 170 (FIG. 1) and/or the application 112, as shown in optional act 218 of FIG. 2. For example, the risk management system 101 may identify current and/or past deviations, identify patterns (e.g., historical trends) based at least partially on the analysis of the operational data. determine predictive models based on the identified patterns and current and/or past deviations, and predict future deviations utilizing the models. Additionally or alternatively, the risk management system 101 may identify current and/or past deviations, identify patterns (e.g., historical trends) based at least partially on the analysis of the operational data, and detect when patterns in current operational data matches the identified patterns.

In one or more embodiments, the risk management system 101 may, based at least partially on the operations logs data and/or the settings data, identify a cause for a given deviation. For example, the risk management system 101 may determine that a deviation was caused by the medical device 170 (FIG. 1). the application 112. the client device 110. based on the operational data (e.g., operational logs and data) and/or identified patterns from the operational data. Additionally, in one or more embodiments, the risk management system 101 may determine that a deviation was caused by one or more settings of an operating system of the client device 110, an operating system of the medical device 170 (FIG. 1), and/or the application 112 of the client device 110. The risk management system 101 may analyze a variety of factors to identify that the deviation was caused by improper device settings. As non-limiting examples. the factors may include: the current device settings of the client device 110, the application 112, and/or the medical device 170

(FIG. 1), whether there were any recent changes to the device settings of the current device settings of the client device 110, the application 112, and/or the medical device 170 (FIG. 1), whether the client device 110 and/or the medical device 170 (FIG. 1) had a recent operating system software update, whether the client device 110 received a notification provided to the client device 110, the time delay between identifying the alarm trigger condition and providing the notification to the client device 110, the quantity of notifications provided to the client device 110, etc.

In various embodiments, in response to determining the cause for one or more deviations, the risk management system 101 of the cloud computing platform 130 may optionally determine a specific response action that needs to be taken by the user 102 (FIG. 1) to correct the one or more deviations, and/or return the medical device 170 (FIG. 1) and/or the application 112 to its intended operation. As is described below; the specific response action may be indicated in a generated notification to the user that includes instructions for performing the specific response (e.g., instructions to adjust one or more improperly configured settings of the operation system of the client device 110, the operating system of the medical device 170 (FIG. 1) and/or the application 112 of the client device 110).

In response to identifying one or more deviations from the intended operation of the medical device 170 (FIG. 1) and/or or the application 112, the risk management system 101 may determine and generate a notification indicating the one or more deviations, as shown in act 220 of FIG. 2. As noted above, in various embodiments, the generated notification may include an indicated response action to correct a deviation.

In various embodiments, determining and generating the notification may include determining (e.g., via the risk management system 101) a level of urgency (e.g., severity) to assign to the notification, as shown in optional act 222 of FIG. 2. In various embodiments, the level of urgency of the notification may be specific to the user 102 (FIG. 1) and may be based on a variety of factors, including the user factor (e.g., the experience level of the user), the sensitivity factor (e.g., sensitivity to disruptions), the significance of the deviation (e.g., a minor deviation that has a minor or no impact on device or system operation. a major deviation that has a major impact on device or system operation and/or indicates device or system malfunctioning), the health of the user (e.g., physiological health (e.g., blood pressure, heart rate, cardiac output, oxygen levels. body temperature, etc.), whether the user uses tobacco products, how often the user exercises, etc.). user conditions (e.g., whether the user is a smoker, how often the user exercises, etc.) information specific to the user's medical condition (e.g., blood glucose level, time of most recent meal, etc.), the severity of the deviation (e.g., minimal and can be resolved by the user, moderate and may need assistance of a healthcare provider, life-threatening and needs immediate assistance of a healthcare provider, etc.).

In various embodiments, the risk management system 101 may assign levels of urgency including low, medium, or high. In various embodiments, low urgency notifications may be warranted for indicating a software update was not properly installed or reminding the user 102 (FIG. 1) of an upcoming insulin delivery that was not properly prompted by one or more of the application 112 or medical device 170, medium urgency notifications may be warranted for warning the user 102 (FIG. 1) of a low quantity of insulin remaining in the medical device 170 (FIG. 1) that was not properly communicated to the user, that a device (e.g., the client device 110) previously lost network connectivity to the medical device 170 (FIG. 1), or that the user 102 (FIG. 1) missed a scheduled insulin dosage, and high urgency notifications may be warranted indicating that the user's blood glucose level is very high or very low and that it was not previously properly indicated to the user or indicating other conditions that may have serious health consequences to the user 102 (FIG. 1) if ignored.

In one or more embodiments, determining and generating the notification may include determining a notification type for the notification, as shown in optional act 224 of FIG. 2. For example, the risk management system 101 of the cloud computing platform 130 may determine a notification type for the notification. For example, the risk management system 101 may determine the type of network to be used to transmit the notification (e.g., cellular data network, voice network, Wi-Fi, etc.) and/or how the notification will be transmitted to and/or provided by the client device 110. For example, the types of notifications may include visual notifications (e.g., text messages, emails, push notifications, lights. etc.). audible notifications (e.g., sounds. phone calls. etc.). and/or haptic notifications (e.g., vibrations, etc.). The notification type may also depend on the type of client device such that the notification type is compatible with the type of client device. Furthermore, the notification type may also depend on an urgency of the notification. For example, a phone call may be warranted for high urgency notifications, while a text message may be sufficient for low urgency notifications.

In various embodiments, the risk management system 101 may generate more than one notification and/or more than one type of notification. Furthermore, the quantity of notifications may also depend on the level of urgency of the notification. For example, a low urgency notification may warrant a single notification of one type (e.g., a single email). A medium urgency notification may warrant multiple notifications that may be of the same type or different types. For example, a medium urgency notification may warrant a text message and a phone call. Additionally, a high urgency notification may warrant multiple notifications and notifications of multiple types. For example, a high urgency notification may warrant multiple of a text message, a phone call, an email. a sound, and/or a vibration. Referring still to act 220, the risk management system 101 may generate the notification according to the above described manners.

In one or more embodiments, the risk management system 101 may further generate a notification for the user's 102 (FIG. 1) healthcare provider, as shown in optional act 225 of FIG. 2. In various embodiments, the notification may include information regarding the one or more deviations. Generating a notification for the user's 102 (FIG. 1) healthcare provider may result in the healthcare provider checking-in with the user 102 (FIG. 1) regarding the one or more deviations, and may increase the safety and effectiveness of the risk management system 101.

The risk management system 101 of the cloud computing platform 130 may provide the notification to the client device 110 and/or the medical device 170 (FIG. 1), as shown in act 226 of FIG. 2. For example, the risk management system 101 of the cloud computing platform 130 may provide the notification to the client device 110 and/or the medical device 170 (FIG. 1) via the network 150 (FIG. 1). Additionally, the notification may optionally include an indication of the above determined level of urgency of the notification, as shown in act 228 of FIG. 2.

The client device 110 may receive the notification from the risk management system 101 of the cloud computing platform 130, as shown in act 230 of FIG. 2. For example. the client device 110 may receive the notification from the risk management system 101 of the cloud computing platform 130 through the network 150 (FIG. 1). The client device 110 may receive the notification via the application 112 and/or via a manner outside of the application 112.

The client device 110 and/or the application 112 may output the notification, as shown in act 232 of FIG. 2. For example, the client device 110 may output the notification by providing a sensory (e.g., haptic, audio, and/or visual) indication of the notification to notify a user. The notification may be output on a visual display and/or speaker on the client device 110 and may cause the client device 110 to vibrate to prompt a user to take an action in response to the notification. In various embodiments, outputting the notification on the client device 110 may include outputting multiple notifications according to the determined level of urgency of the notification described above.

In various embodiments, the client device 110 may detect a user action responsive to the notification, as shown in optional act 234 of FIG. 2. For example, the client device 110 may detect that the user 102 (FIG. 1) has taken an action acknowledging the notification and/or performed a response action responsive to the notification and/or identified deviation. In various embodiments. the user action may include the user 102 (FIG. 1) interacting with a graphical user interface of the medical device 170 (FIG. 1), the client device 110, and/or the application 112 displaying the notification (e.g., selecting an acknowledgement button or sending a reply text). In other embodiments, the user action may include the user delivering medication, adjusting a setting, initiating charging of a battery, or any other response action described herein.

The client device 110 may provide a data package to the risk management system 101 of the cloud computing platform 130. as shown in optional act 236 of FIG. 2. The data package may indicate information related to the user action. The application 112 may also provide a data package to the risk management system 101 of the cloud computing platform 130 via the client device 110 and/or network 150 (FIG. 1).

The risk management system 101 of the cloud computing platform 130 may receive the data package from the client device 110, as shown in optional act 238 of FIG. 2. Based on the data package, risk management system 101 of the cloud computing platform 130 may determine whether an appropriate response action has been made relative to deviations indicated in notification (act 220), as shown in optional act 240 of FIG. 2.

In response to detecting whether an appropriate response action has been performed. the risk management system 101 may terminate operations related to the identified deviations.

Conversely, responsive to determining that the recommended response action has still not been performed properly, the risk management system 101 of the cloud computing platform 130 may determine and generate another notification indicating the one or more deviations according to any of the manners described above in regard to act 220, as shown in optional act 242 of FIG. 2. Additionally, the risk management system 101 may determine a level of urgency (e.g., severity) of the another notification via any of the manners described above in regard to act 222, as shown in optional act 244 of FIG. 2. In various embodiments, the another notification may be determined to have a different level of urgency than the notification. For example, the notification may have a first level of urgency and the another notification may have a second level of urgency. Additionally. the urgency level of the another notification (e.g., second level of urgency) may be higher or more urgent than a level of urgency of the notification (e.g., the first level of urgency). For example, if the notification had an urgency level of low, the another notification may have an urgency level of medium or high.

In one or more embodiments, the risk management system 101 may determine a notification type of the another notification via any of the manners described above in regard to act 224, as shown in optional act 246 of FIG. 2. In various embodiments, the risk management system 101 may change the type of the another notification relative to the previously provided notification (e.g., a first notification) if the type of the previously provided notification resulted in no response within a given time frame.

In one or more embodiments. the risk management system 101 may generate another notification for the user's 102 (FIG. 1) healthcare provider via any of the manners described above in regard to act 240, as shown in optional act 247 of FIG. 2. The notification for the user's 102 (FIG. 1) healthcare provider may include information regarding the one or more deviations and that the user 102 (FIG. 1) has not taken a recommend response action, and may result in the healthcare provider checking-in with the user 102 (FIG. 1) regarding the one or more deviations. Generating the another notification for the user's healthcare provider when the user 102 (FIG. 1) appears to be unresponsive to the notification may increase the safety of the user and effectiveness of the risk management system 101 (FIG. 1).

The risk management system 101 of the cloud computing platform 130 may provide the another notification to the client device 110 and/or the medical device 170 (FIG. 1), as shown in optional act 248 of FIG. 2. Additionally, the another notification may indicate the determined level of urgency of the another notification, as shown in act 250 of FIG. 2.

In one or more embodiments, the client device 110 may receive the another notification from the risk management system 101 cloud computing platform 130 through the network 150 (FIG. 1), as shown in optional act 252 of FIG. 2.

In various embodiments, the risk management system 101 of the cloud computing platform 130 and/or the client device 110 may repeat one or more acts from 232-252 of FIG. 2 until an appropriate response action has been made (e.g., by a user, or a user's healthcare provider).

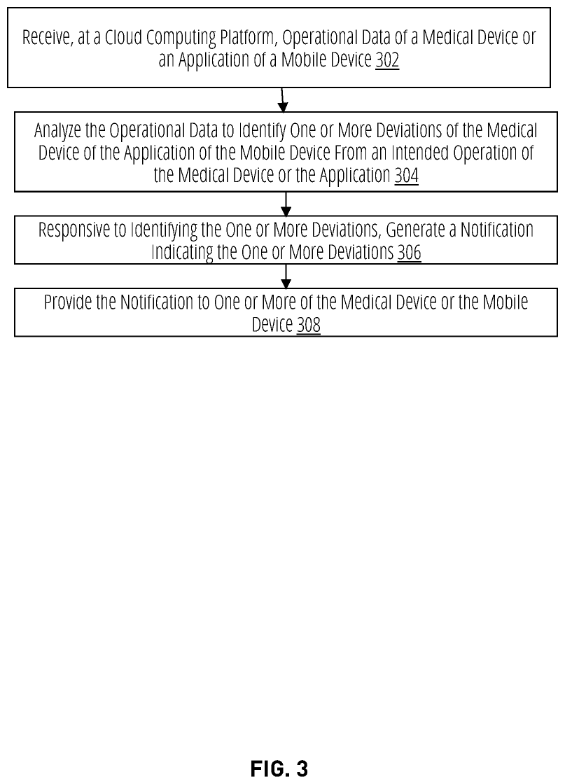

FIG. 3 illustrates a method 300, in accordance with one or more embodiments of the disclosure, that a risk management system may utilize to analyze operation of an application and/or a medical device and to notify a user of a deviation from an intended operation of the application and/or the medical device. As described herein, the method 300 may be performed by the system and/or one or more devices of illustrated in FIG. 1, including, for example, the cloud computing platform 130, the risk management system 101, the client device 110, the application 112, the medical device 170, the network 150, and/or the user 102.

The method 300 may involve, for example, receiving, at a cloud computing platform, operational data of a medical device or an application of a client device, as illustrated at act 302. Receiving the operational data may include any of the actions described above in regard to acts 204 and 205 of FIG. 2.

The operational data may be analyzed (e.g., by the risk management system of the cloud computing platform) to identify one or more deviations of the medical device and/or the application of the client device from an intended operation of the medical device and/or the application, as illustrated at act 304. Analyzing the operational data may include any of the actions described above in regard to acts 206 through 218 of FIG. 2.

In response to identifying the one or more deviations, a notification may be generated (e.g., by the cloud computing platform) indicating the one or more deviations, as illustrated at act 306. Generating the notification may include any of the actions described above in regard to acts 220 through 225 of FIG. 2.

The notification may be provided to one or more of the medical devices or the client device, as illustrated at act 308. Providing the notification to the medical device and/or the client device may include any of the actions described above in regard to acts 226 and 226 of FIG. 2.

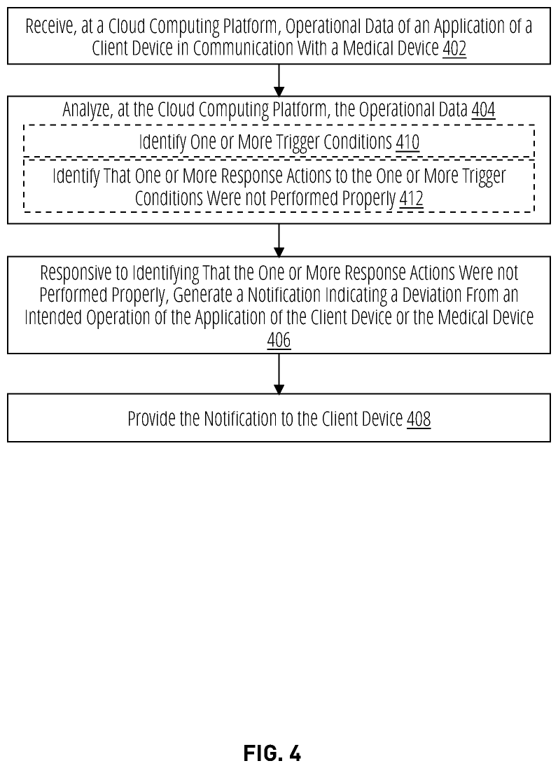

FIG. 4 illustrates a method 400 in accordance with one or more embodiments of the disclosure that a risk management system may utilize to analyze operation of an application and/or a medical device, and may notify a user of a deviation from an intended operation of the application and/or the medical device. As described herein, the method 400 may be performed by the system and/or one or more devices of illustrated in FIG. 1, including, for example, the cloud computing platform 130, the risk management system 101, the client device 110, the application 112, the medical device 170, the network 150, and/or the user 102.

The method 400 may involve, for example, receiving, at a cloud computing platform, operational data of an application of a client device in communication with a medical device, as illustrated at act 402. Receiving the operational data may include any of the actions described above in regard to acts 204 and 205 of FIG. 2.

The operational data may be analyzed at the cloud computing platform (e.g., by the risk management system), as illustrated at act 404. Analyzing the operational data at the cloud computing platform may include identifying one or more trigger conditions, as illustrated at optional act 410. Analyzing the operational data at the cloud computing platform may also include identifying that one or more response actions to the one or more trigger conditions were not properly performed, as illustrated at optional act 412. Analyzing the operational data may include any of the actions described above in regard to acts 206 through 218 of FIG. 2.

In response to identifying that one or more response actions were not performed properly, a notification may be generated (e g, by the cloud computing platform) indicating a deviation from an intended operation of the application of the client device or the medical device, as illustrated at act 406. Generating the notification may include any of the actions described above in regard to acts 220 through 225 of FIG. 2.

The notification may be provided to the client device, as illustrated at act 408. Providing the notification to the medical device and/or the client device may include any of the actions described above in regard to acts 226 and 226 of FIG. 2.

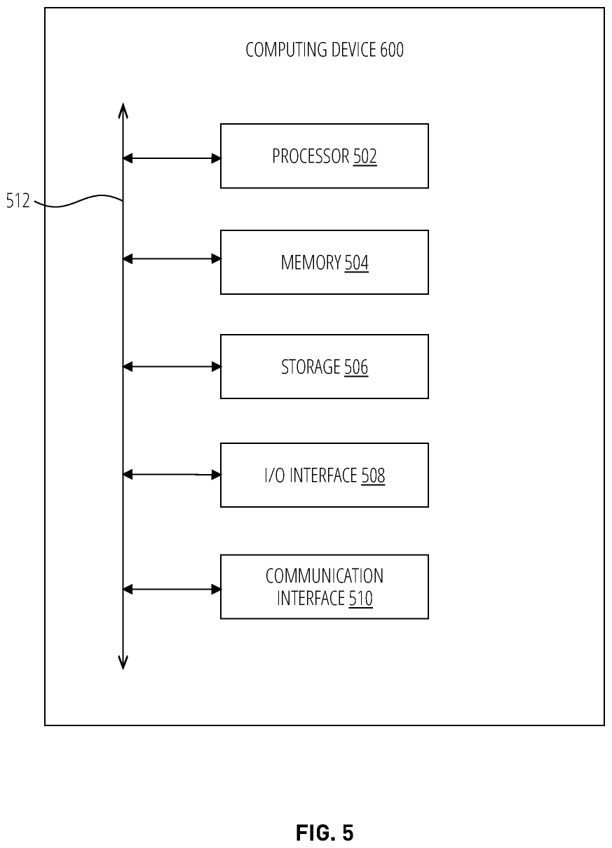

FIG. 5 illustrates a block diagram of an example computing device 500 that may be configured to perform one or more of the processes described above and may be included in the risk management system 101 (FIG. 1). One will appreciate that one or more computing devices such as the computing device 500 may also be included within the cloud computing platform 130 (FIG. 1), the client device 110 (FIG. 1), and/or the medical device 170 (FIG. 1).

As shown by FIG. 5, the computing device 500 may comprise a processor 502, a memory 504. a storage device 506, an I/O interface 508, and a communication interface 510, which may be communicatively coupled by way of a communication infrastructure. While an example computing device 500 is shown in FIG. 5, the components illustrated in FIG. 5 are not intended to be limiting. Additional or alternative components may be used in other embodiments. Furthermore, in certain embodiments, the computing device 500 may include fewer components than those shown in FIG. 5. Components of the computing device 500 shown in FIG. 5 will now be described in additional detail.

In one or more embodiments. the processor 502 includes hardware for executing instructions, such as those making up a computer program. As an example, and not by way of limitation, to execute instructions, the processor 502 may retrieve (or fetch) the instructions from an internal register, an internal cache. the memory 504, or the storage device 506 and decode and execute them. In one or more embodiments, the processor 502 may include one or more internal caches for data, instructions, or addresses. As an example, and not by way of limitation, the processor 502 may include one or more instruction caches, one or more data caches, and one or more translation look aside buffers (TLBs). Instructions in the instruction caches may be copies of instructions in the memory 504 or the storage 506.

The computing device 500 includes memory 504, which is coupled to the processor(s) 502. The memory 504 may be used for storing data, metadata, and programs for execution by the processor(s). The memory 504 may include one or more of volatile and non-volatile memories. such as Random-Access Memory (“RAM”). Read-Only Memory (“ROM”), a solid state disk (“SSD”), Flash, Phase Change Memory (“PCM”), or other types of data storage. The memory 504 may be internal or distributed memory.

The computing device 500 includes a storage device 506 that includes storage for storing data or instructions. As an example, and not by way of limitation, storage device 506 may comprise a non-transitory storage medium described above. The storage device 506 may include a hard disk drive (HDD), a floppy disk drive, Flash memory, an optical disc, a magneto-optical disc, magnetic tape. or a Universal Serial Bus (USB) drive or a combination of two or more of these. The storage device 506 may include removable or non-removable (or fixed) media. where appropriate. The storage device 506 may be internal or external to the computing device 500. In one or more embodiments, the storage device 506 is non-volatile, solid-state memory. In other embodiments, the storage device 506 includes read-only memory (ROM). Where appropriate, this ROM may be mask programmed ROM, programmable ROM (PROM), erasable PROM (EPROM), electrically erasable PROM (EEPROM), electrically alterable ROM (EAROM), or Flash memory or a combination of two or more of these.

The computing device 500 also includes one or more input or output (“I/O”) devices/interfaces 508, which are provided to allow a user to provide input to, receive output from. and otherwise transfer data to and receive data from computing device 500. The I/O devices/interfaces 508 may include a mouse, a keypad or a keyboard, a touch screen, a camera, an optical scanner, network interface, modem, other known I/O devices or a combination of such I/O device/interfaces. The touch screen may be activated with a stylus or a finger.

The I/O devices/interfaces 508 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 508 is configured to provide graphical data to a display for presentation to a user. The graphical data may be representative of one or more graphical user interfaces and/or any other graphical content as may serve a particular implementation.

The computing device 500 may further include a communication interface 510. The communication interface 510 may include hardware, software, or both. The communication interface 510 may provide one or more interfaces for communication (such as, for example, packet-based communication) between the computing device 500 and one or more other computing devices or networks. As an example, and not by way of limitation, the communication interface 510 may include a network interface controller (NIC) or network adapter for communicating with an Ethernet or other wire-based network or a wireless NIC (WNIC) or wireless adapter for communicating with a wireless network, such as a WI-FI. The computing device 500 may further include a bus 512. The bus 512 may comprise hardware, software, or both that couples components of computing device 500 to each other.

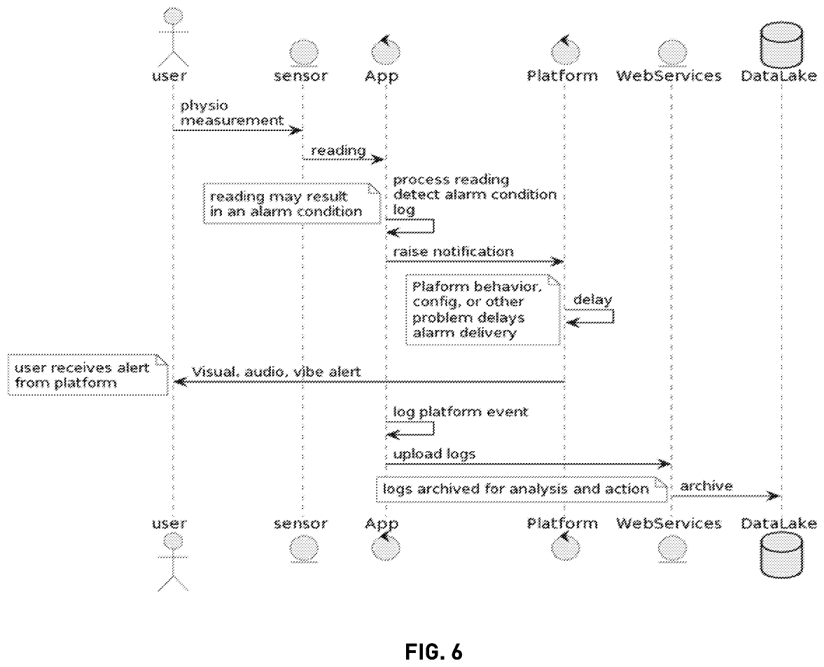

FIG. 6 illustrates a diagram of an example flow for a system that maintains a catalog of platform dependent features, settings, and uses configurable values, and log tags for the same.

Platform-dependent features, settings and other configurations for the features, and user configured values of the same, are compiled and maintained in an electronic catalog. In some cases, an importance rating may be assigned to one or more of the features, settings, configurations, or values, and only those that are above a threshold importance are included as an element of the electronic catalog. For each element of the catalog, a trace tag is added to the electronic catalog. The trace tag is the name or other unique, alpha-numerical identifier utilized to identify the element in the operations logs. The elements in the electronic catalog are searchable within the electronic catalog using the trace tag as the search term. When operations logs are uploaded via a webservice to a data lake where they are archived and the data therein may be processed for analysis.

The flow illustrated by FIG. 6 is a non-limiting example of uploading, archiving and analysis. A physiological (physio) measurement about a user is captured by a sensor (e.g., a physiological sensor, without limitation). The value(s) of the physiological measurement is read by an application (e.g., software application, without limitation) executing on a client device (e.g., a mobile device such as a smartphone, without limitation). The process executed by the application that reads the value of the physiological measurement detects, triggers, initiates, or causes an alarm condition and that a log about the alarm condition is generated. The application, either automatically or at the instruction of the user, raises a notification (e.g., requests a visual, audio, or haptic alarm or alert be delivered to the user, without limitation) to the client device platform (e.g., a mobile operating system of a smartphone such as Android or iOS, without limitation). Delivery of the notification is delayed for some non-momentary time duration. After the non-momentary time duration, the platform delivers the notification to the user.

A desired time of delivery may be preset in the application or via the platform service with which the notification is raised by the application. The desired time of delivery may be used as a threshold by the application to detect a delay condition. Logs about the platform behavior, configurations, conditions, states are created automatically by the platform, by the platform in response to a request by the application, or by the application (e.g., using trace data generated by the platform and requested by the application, without limitation). In one or more examples, the logs are generated upon detection of the delay condition, and may represent a snapshot of the platform at that moment in time. Additionally or alternatively, trace is generated continuously and it is captured for a time period that generally correspond to when the notification was raised and the delay condition detected. The boundaries of the range of time for which trace is compiled for the log may be preset and vary depending on the condition. For example, trace may be gathered for 1 to 5 minutes before the delay condition was detected and 1 to 5 minutes after the delay condition was detected. In one or more examples, the operations logs include at least some of the trace tags that were added to the electronic catalog.

The logs are sent (e.g., uploaded via the Internet, without limitation) to a webservice using, e.g., the local communication services of the platform. The logs may be sent automatically, upon request by the webservice, or when the application next synchronizes with the webservice. The webservice archives the operations logs for analysis and action with a DataLake. Trace tags in the operations logs may be utilized to search the electronic catalog to identify elements that are the subject of the traces and assist with the analysis and actions, if any.

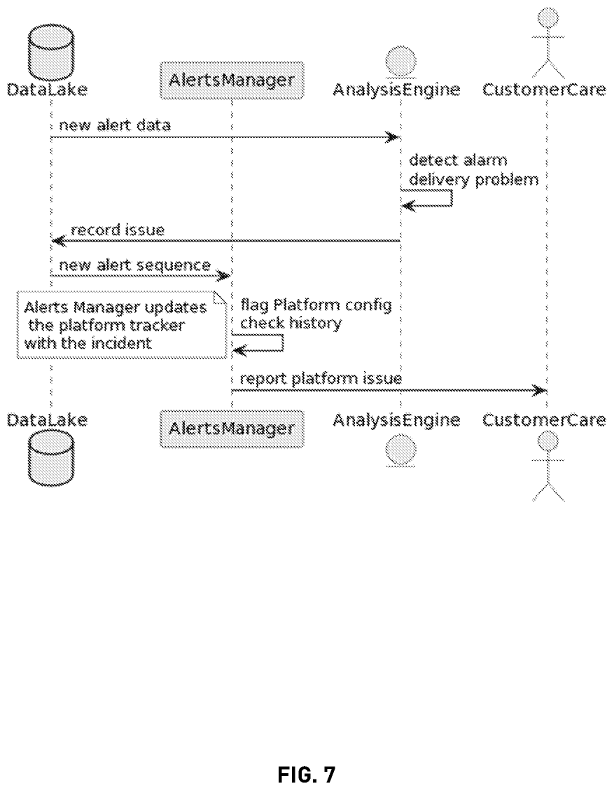

FIG. 7 illustrates a diagram of an example flow for a system that analyzes operations logs archived in a DataLake, in accordance with one or more examples. By way of non-limiting examples, the operations logs discussed with respect to the example illustrated by FIG. 7 may be the ones uploaded in the flow illustrated by FIG. 6.

An Analysis Engine analyzes the archived log data, identifies relevant alert events, and analyzes relationships between application and platform events. For example, if a log shows an alarm trigger condition was detected by the application at a given point in time but the platform-delivered alarm action is delayed, one can infer compromised performance.

An Alerts Manager tracks platform configurations and events. For a given configuration, compare relevant trace events with the platform catalog entry associated with the configuration. Results from this passive compatibility analysis may be used to infer critical features compatibility for a given configuration. The alerts manager may notify any system or party (e.g., a customer care system, a field services system, a clinical application specialist system, a support entity system. other support system without limitation) of such events.

The Alerts Manager may use in-app messaging (e.g., an in-application messaging service of the application on the client device, without limitation) or other communication means to notify the user of an issue and, optionally, indicate corrective actions.

An application user for whom such a problem is identified may, as needed, take prompt action to remediate. But this is after the fact-and some notifications sent via in-app messaging may be preemptive or educational. to educate and remind a user of the potential of such problems which are undetectable by the application before they occur.

An extension to the alerts manager may raise a ‘smart warning’ which is displayed intermittently, based on cloud-based logic, to tailor the delivery of important reminders while reducing annoyance and alarm fatigue. A cloud-based functionality may continuously monitor each operations log, and detect smart warning conditions represented in one or more operations logs using predetermined criteria. Conditions meeting warning criteria will result in the creation of a record specific to that user (a “trigger record”). When the user's application next synchronizes with web services it will receive the trigger record. The application will deliver the messaging to the user. The alerts manager may also provide, during next synchronization, messages with tailored information and reinforcement for users new to the system or experiencing more alarm conditions. This tailored information may be provided via in-app messaging to the user.

The analysis engine may detect patterns of alarm triggers or other critical events. An individual experiencing more critical events will be more sensitive to disruptions in the application's ability to perform critical tasks. More frequent warnings to the user may be appropriate. A user new to the application may be reminded more often than a more experienced user. The Alerts Manager may in response to being notified of such a pattern, may send one or more messages that include such warnings and reminders to the user via the in-app messaging service.

The foregoing specification is described with reference to specific example embodiments thereof. Various embodiments and aspects of the disclosure are described with reference to details discussed herein, and the accompanying drawings illustrate the various embodiments. The description above and drawings are illustrative and are not to be construed as limiting. Numerous specific details are described to provide a thorough understanding of various embodiments.

The additional or alternative embodiments may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

What is claimed is:

1. A method, comprising:
receiving, at a cloud computing platform, operational data of a medical device or an application of a client device, the application being associated with and utilized to at least partially to operate the medical device;
analyzing the operational data to identify one or more deviations from an intended operation of the medical device or the application;
responsive to identifying the one or more deviations, generating a notification indicating the one or more deviations; and
providing the notification to one or more of the medical devices or the client device.

2. The method of claim 1, further comprising receiving, at the cloud computing platform, a response action responsive to the notification from the client device.

3. The method of claim 1, further comprising:
determining that no response action has been made;
responsive to determining that no response action has been made according to selected criteria, generating another notification indicating the one or more deviations; and
providing the another notification to one or more of the medical devices or the client device.

4. The method of claim 3, wherein providing the notification comprises providing the notification having a first level of urgency, and wherein providing the another notification comprises providing the another notification having a second level of urgency.

5. The method of claim 4, wherein providing the another notification comprises providing a more urgent notification than providing the notification.

6. The method of claim 1, wherein analyzing the operational data to determine one or more deviations comprises identifying historical trends of the operational data and predicting one or more future deviations from the intended operation of the medical device or the application based on the identified historical trends.

7. The method of claim 1, wherein analyzing the operational data to determine one or more deviations comprises analyzing the operational data to determine one or more of a past or a current deviation from the intended operation of the medical device or the application.

8. The method of claim 1, wherein providing the notification to one or more of the medical devices or the client device comprises providing one or more of a push notification or an audible notification.

9. The method of claim 1, wherein analyzing the operational data comprises identifying that the one or more deviations originate from one or more of improper settings of an operating system of the client device, an operating system of the medical device, or the application of the client device.

10. The method of claim 9, further comprising generating one or more instructions to adjust settings of the one or more of improper settings of an operating system of the client device, an operating system of the medical device, or the application of the client device.

11. The method of claim 1, wherein generating a notification comprises generating the notification to indicate a severity of the one or more deviations from the intended operation.

12. A method of determining operational performance of an application, the method comprising:
receiving, at a cloud computing platform, operational data of the application of a client device in communication with a medical device:
analyzing, at the cloud computing platform, the operational data comprising:
identifying one or more trigger conditions; and
identifying that one or more response actions to the one or more trigger conditions were not performed properly:
responsive to identifying that the one or more response actions were not performed properly, generating a notification indicating a deviation from an intended operation of the application of the client device or the medical device: and
providing the notification to the client device.

13. The method of claim 12, wherein identifying that one or more response actions to the one or more trigger conditions were not performed properly comprises identifying one or more response actions that were delayed before performance.

14. The method of claim 12, wherein identifying that one or more response actions to the one or more trigger conditions were not performed properly comprises identifying one or more response actions that were not performed at all or only partially performed.

15. The method of claim 12, analyzing the operational data comprises analyzing operational logs.

16. The method of claim 12, wherein analyzing the operational data comprises analyzing the operational data via one or more machine learning techniques.

17. The method of claim 12, wherein the operational data is received via a synchronizing event between the application of the client device and the cloud computing platform.

18. The method of claim 12, wherein analyzing the operational data further comprises identifying patterns of triggering conditions.

19. The method of claim 12, further comprising determining a frequency at which to provide notifications to the client device regarding incorrect operations.

20. The method of claim 19, wherein determining a frequency at which to provide notifications to the client device regarding incorrect operations comprises providing more frequent notifications in response to the client device being associated with a less experienced user, and providing less frequent notifications in response to the client device being associated with a more experienced user.

21. A cloud computing platform comprising:
a processor; and
a memory storing instructions thereon that, when executed by the processor, cause the cloud computing platform to:
receive, from a mobile device, operational data of a medical device or an application of the mobile device:
analyze the operational data to identify one or more deviations from an intended operation of the medical device or the application:
responsive to identifying the one or more deviations, generate a notification indicating the one or more deviations: and
provide the notification to the mobile device one or more times according to a determined frequency.

22. The cloud computing platform of claim 21, wherein the memory comprises additional instructions thereon that, when executed by the processor, cause the cloud computing platform to provide more frequent notifications in response to the mobile device being associated with a less experienced user, and provide less frequent notifications in response to the mobile device being associated with a more experienced user.

23. The cloud computing platform of claim 21. wherein the memory comprises additional instructions thereon that. when executed by the processor. cause the cloud computing platform to analyze the operational data via one or more machine learning techniques.
